(19) United States
(12) Patent Application Publication
(10) Pub. No.: US 2025/0154094 A1
(43) Pub. Date: May 15, 2025

(54) NOVEL CURCUMIN DERIVATIVE WITH ENHANCED ANTIOXIDANT PROPERTIES

(71) Applicant: KING FAISAL UNIVERSITY, AL-AHSA (SA)

(72) Inventors: IMENE BAYACH, AL-AHSA (SA); NADEEM S. SHEIKH, AL-AHSA (SA)

(21) Appl. No.: 18/508,977

(22) Filed: Nov. 14, 2023

(51) Int. Cl. C07C 225/16; A61K 8/41

(52) U.S. Cl. CPC C07C 225/16; A61K 8/415

(57) ABSTRACT

A structural modification of curcumin designed to increase its antioxidant activity is provided. The approach involves the substitution of one methoxy group (OCH₃) by methyl (CH₃) and the substitution of one hydroxy group (OH), at the other end of the molecule from the substituted methoxy group, with an amino group (NH₂), resulting in the formation of a new structure. Based on quantum calculations, this modification may enhance the antioxidant properties of curcumin and its ability to scavenge free radicals.

### BACKGROUND

### 1. Field

The present disclosure relates to a new antioxidant molecule, a curcumin derivative that may enhance the antioxidant properties of curcumin and its ability to scavenge free radicals.

### 2. Description of the Related Art

Curcumin, a natural polyphenol derived from turmeric, has gained significant attention for its antioxidant properties and potential health benefits. However, Curcumin has certain limitations, including its relatively low Bond Dissociation Energy (BDE) of 80.9, which affects its overall antioxidant efficacy.

Numerous derivatives of curcumin have been proposed to solve this problem. However, to date, no viable curcumin derivatives have been developed.

Thus, the development of new antioxidants, and particularly to new curcumin derivatives, solving the aforementioned problems are desired.

### SUMMARY

The present subject matter relates to the structural modification of curcumin to increase its antioxidant activity. The approach involves the substitution of one methoxy group (OCH₃) by methyl (CH₃) and the substitution of one hydroxy group (OH), at the other end of the molecule from the substituted methoxy group, with an amino group (NH₂), resulting in the formation of a new structure. Based on quantum calculations, this modification may enhance the antioxidant properties of curcumin and its ability to scavenge free radicals.

In an embodiment, the present subject matter relates to a curcumin derivative compound having the formula:

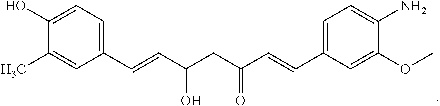 [SMILES: COc1cc(/C=C/C(=O)CC(O)/C=C/c2ccc(O)c(C)c2)ccc1N]

In another embodiment, the present subject matter relates to a pharmaceutical composition, comprising the curcumin derivative compound and a pharmaceutically acceptable carrier.

In a further embodiment, the present subject matter relates to a cosmetic composition, comprising the curcumin derivative compound and a cosmetically acceptable carrier.

In additional embodiments, the present subject matter relates to methods of promoting an antioxidant activity in a subject, promoting skin health in a subject, and/or protecting skin against oxidative damage in a subject, the methods comprising administering to a subject in need thereof the pharmaceutical composition and/or the cosmetic composition as described herein.

These and other features of the present subject matter will become readily apparent upon further review of the following specification.

### DETAILED DESCRIPTION OF THE PREFERRED EMBODIMENTS

The following definitions are provided for the purpose of understanding the present subject matter and for construing the appended patent claims.

### Definitions

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl,” as defined herein.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

“Subject” as used herein refers to any animal classified as a mammal, including humans, domestic and farm animals, and zoo, sports, and pet companion animals such as household pets and other domesticated animals such as, but not limited to, cattle, sheep, ferrets, swine, horses, poultry, rabbits, goats, dogs, cats and the like.

“Patient” as used herein refers to a subject in need of treatment of a condition, disorder, or disease, such as a disorder or disease having an oxidative component.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In an embodiment, the present subject matter relates to a curcumin derivative compound having the formula:

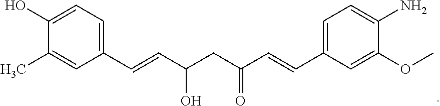 [SMILES: COc1cc(/C=C/C(=O)CC(O)/C=C/c2ccc(O)c(C)c2)ccc1N]

In this regard, the present curcumin derivative involves the substitution of one methoxy group (OCH₃) by methyl (CH₃) and the substitution of one hydroxy group (OH), at the other end of the molecule from the substituted methoxy group, with an amino group (NH₂), resulting in the formation of a new structure. That is, the present curcumin derivative incorporates specific functional groups (—OH, —CH₃, —OCH₃, and —NH₂), as can be seen from the following comparison between curcumin and the present curcumin derivative:

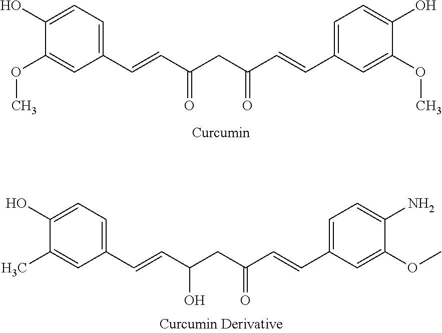 [SMILES: COc1cc(/C=C/C(=O)CC(=O)/C=C/c2ccc(O)c(OC)c2)ccc1O ; COc1cc(/C=C/C(=O)CC(O)/C=C/c2ccc(O)c(C)c2)ccc1N]

These substitutions were made to enhance the new derivative's Bond Dissociation Energy (BDE). In this regard, Density Functional Theory (DFT) calculations were performed to determine the BDE values for curcumin and the derivative described herein, as well as other, less active derivatives. Such DFT calculations are well known in the art to be accurate in predicting antioxidant activity of compounds.

In this regard, the present curcumin derivative has a BDE value of 78.4, representing a significant improvement over the BDE of 80.9 for curcumin, thus indicating superior antioxidant capabilities and activities for the present curcumin derivative. Accordingly, the present curcumin derivative can enhance the antioxidant properties of curcumin and its ability to scavenge free radicals, thereby making the present curcumin derivative more effective in combating oxidative stress and free radical damage, among other enhanced activities. Further, the lower BDE value for the present curcumin derivative means it has enhanced stability, allowing for a longer shelf-life and improved bioavailability.

Accordingly, in an embodiment, the curcumin derivative compound has a bond dissociation enthalpy (BDE) of about 78 kcal/mol.

In another embodiment, the present subject matter relates to a pharmaceutical composition, comprising the curcumin derivative compound and a pharmaceutically acceptable carrier.

In a further embodiment, the present subject matter relates to a cosmetic composition, comprising the curcumin derivative compound and a cosmetically acceptable carrier.

Non-limiting examples of suitable excipients, carriers, or vehicles useful herein include liquids such as water, saline, glycerol, polyethylene glycol, hyaluronic acid, ethanol, and the like. Suitable excipients for nonliquid formulations are also known to those of skill in the art. A thorough discussion of pharmaceutically acceptable excipients and salts useful herein is available in Remington's Pharmaceutical Sciences, 18th Edition. Easton, Pa., Mack Publishing Company, 1990, the entire contents of which are incorporated by reference herein.

The present compound would typically be administered at a therapeutically or pharmaceutically effective dosage, or at a cosmetically acceptable dosage, which would be expected to be lower than the therapeutically or pharmaceutically effective dosage. Administration of the compounds or compositions thereof can be by any method that delivers the compounds systemically and/or locally. These methods include oral routes, parenteral routes, intraduodenal routes, and the like.

While human dosage levels have yet to be optimized for the present compounds, generally, a daily dose is from about 0.01 to 10.0 mg/kg of body weight, for example about 0.1 to 5.0 mg/kg of body weight. The precise effective amount will vary from subject to subject and will depend upon the species, age, the subject's size and health, the nature and extent of the condition being treated, recommendations of the treating physician, and the therapeutics/cosmetics or combination of therapeutics/cosmetics selected for administration. The subject may be administered as many doses as is required to reduce and/or alleviate the signs, symptoms, or causes of the disease or disorder in question, or bring about any other desired alteration of a biological system.

In employing the present compound, any pharmaceutically or cosmetically acceptable mode of administration can be used with other acceptable excipients, including solid, semi-solid, liquid or aerosol dosage forms, such as, for example, tablets, capsules, powders, liquids, suspensions, suppositories, aerosols or the like. In an embodiment, the dosage form is likely to be a topical dosage form, such as a cream, lotion, ointment, gel, foam, aerosol, or the like. In such an embodiment, the topical dosage form is likely to be applied to an area of a patient's skin to be treated.

In additional embodiments, the present subject matter relates to methods of promoting an antioxidant activity in a subject, promoting skin health in a subject, and/or protecting skin against oxidative damage in a subject, the methods comprising administering to a subject in need thereof the pharmaceutical composition and/or the cosmetic composition as described herein.

In this regard, the increased antioxidant capacity of the present curcumin derivative may lead to enhanced health benefits, including but not limited to improved anti-inflammatory, anti-aging, and neuroprotective effects.

Generally, depending on the intended mode of administration, the composition will contain about 0.1% to 90%, for example about 0.5% to 50%, by weight of the present compound, the remainder being suitable excipients, carriers, etc.

The present compound is expected to have valuable pharmaceutical and cosmetic properties, which makes it commercially utilizable. Accordingly, the present subject matter further relates to use of the present compound for the treatment of diseases, disorders, or conditions having oxidative activity.

Accordingly, the present curcumin derivative has a wide range of potential applications, including but not limited to:
Use in pharmaceutical formulations for the treatment of oxidative stress-related diseases.
Use in nutraceutical supplements targeting antioxidant support and overall health.
Use in cosmetic and skincare products to combat skin aging and damage caused by free radicals.
Use as a food additive for enhancing the shelf life and nutritional value of various products.

It is to be understood that the curcumin derivative compound is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

We claim:

1. A curcumin derivative compound having the formula:

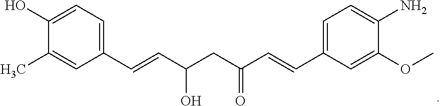 [SMILES: COc1cc(/C=C/C(=O)CC(O)/C=C/c2ccc(O)c(C)c2)ccc1N]

2. The curcumin derivative compound of claim 1, having a bond dissociation enthalpy (BDE) of about 78 kcal/mol.

3. A pharmaceutical composition, comprising the curcumin derivative compound of claim 1 and a pharmaceutically acceptable carrier.

4. A cosmetic composition, comprising the curcumin derivative compound of claim 1 and a cosmetically acceptable carrier.

5. A method of promoting an antioxidant activity in a subject, the method comprising administering to a subject in need thereof the pharmaceutical composition of claim 3.

6. A method of promoting an antioxidant activity in a subject, the method comprising administering to a subject in need thereof the cosmetic composition of claim 4.

7. A method of promoting skin health in a subject, the method comprising administering to a subject in need thereof the pharmaceutical composition of claim 3.

8. A method of promoting skin health in a subject, the method comprising administering to a subject in need thereof the cosmetic composition of claim 4.

9. A method of protecting skin against oxidative damage in a subject, the method comprising administering to a subject in need thereof the pharmaceutical composition of claim 3.

10. A method of protecting skin against oxidative damage in a subject, the method comprising administering to a subject in need thereof the cosmetic composition of claim 4.
